(19) United States
(12) Patent Application Publication
(10) Pub. No.: US 2004/0055033 A1
(43) Pub. Date: Mar. 18, 2004

(54) Multiple disease resistant cucumber inbred line

(76) Inventors: Henry M. Munger, Ithaca, NY (US); Margaret M. Jahn, Lansing, NY (US)

Correspondence Address: Michael L. Goldman, Esq., NIXON PEABODY LLP, Clinton Square, P.O. Box 31051, Rochester, NY, 14603-1051, US

(21) Appl. No.: 10/246,197

(22) Filed: Sep. 18, 2002

(51) Int. Cl. A01H 1/00; C12N 15/82; A01H 5/00; C12N 5/04

(52) U.S. Cl. 800/279; 800/307; 435/419

(57) ABSTRACT

The present invention relates to a multiple disease resistant inbred cucumber seed. The present invention also relates to a method of producing a multiple disease resistant inbred cucumber seed, via the backcross method, combining resistance to target leafspot disease in one C. sativus L. cucumber cultivar with resistant to zucchini yellow mosaic virus, watermelon mosaic virus, and papaya ringspot virus in a second C. sativus L. cucumber cultivar. The present invention is further directed to plant seeds, plants, pollen, and ovules produced by carrying out this method, as well as cucumber plants and seeds produced by using the inbred multiple disease resistant cucumber of the present invention as a parent in a breeding program.

This invention was developed with government funding under Hatch Grant, No. 149485. The U.S. Government may have certain rights.

### FIELD OF THE INVENTION

The present invention relates to C. sativus L. inbred seeds and plants having multiple disease resistance, methods of producing multiple disease resistance C. sativus L seeds and plants, and multiple disease resistant seeds and plants produced by using the inbred C. sativus L. as a parent in a breeding program.

### BACKGROUND OF THE INVENTION

Cucumbers originated in India where they have been cultivated for 3000 years. The cucumber (Cucumis sativus L.) is a member of the Cucurbitaceae family, also known as the gourd family. This family consists of more than 100 genera, but only three are of commercial importance in the United States. These are Cucumis (cucumber and muskmelon), Citrullis (watermelon), and Cucurbita (pumpkin and squash).

Cucurbits are generally annuals that are extremely intolerant of cold weather. They are grown mainly for their fruits, which are derived from a single ovary containing many ovules or seeds. In some parts of the world, flowers and leaves of some species are also used for food. Among vegetable crops, the cucurbits are somewhat different in that they bear different kinds of flowers on the same plant. In most cucurbits, the pollen is rather sticky and heavy, and pollination by insects is required. Cucumbers are both a leading commercial crop and a popular home garden vegetable. Cucumber cultivars are usually classified according to their intended use as fresh market slicers, pickles, or greenhouse cucumbers.

The major portion of the commercial cucumber crop in the U.S. is processed as pickles, with Michigan, North Carolina, Texas, California, and Wisconsin the leading states for pickle production. Processing cucumber yields average about 5 tons per acre in the U.S., and range from about 4 tons per acre in the east to over 18 tons per acre in California. Approximately 40% of the pickling crop is fresh-packed, 15% is refrigerated, and 40-45% is brined. Cucumbers for pickling are usually grown under contract, so profits are directly related to yield per acre.

Florida, the Carolinas, and Texas lead in fresh market production. Average yields for fresh market slicers picked by hand range from 3 tons per acre in parts of the eastern U.S. to over 16 tons per acre in California; top yields can be much higher. Fresh market cucumbers produced early in the marketing season are a high-value crop in most areas of the U.S.

Cucurbits are affected by a large number of diseases caused by bacteria, fungi, and viruses. Among the most common cucumber diseases are angular leafspot disease, caused by the bacterium Pseudomonas lachrymans; cucumber mosaic virus (“CMV”), which is found in many areas of the country, overwinters on seeds and roots, and is spread by insects; and scab, caused by the fungus Cladosporium cucumerinum, which produces sunken, dark brown spots on the fruits. In addition, cucumbers may be infected by target leafspot disease (“TLS”) caused by Corynespora cassiicola, zucchini yellow mosaic virus (“ZYMV”), watermelon mosaic virus (“WMV”), papaya ringspot virus (“PRSV”), downy mildew, anthracnose, root knot (caused by nematodes), damping-off, powdery mildew, Alternaria and Cercospora leaf blights, gummy stem blight, and black rot. Viral diseases are often the most destructive and the most difficult to control.

Disease control is critical in the large-scale production of high quality slicing cucumbers. A preventative program generally combines the use of cultural practices, genetic resistance, and chemical control to limit the establishment, spreading, and survival of cucumber pathogens. However, cultural practices require frequent crop rotation, optimal soil conditions, and exacting planting conditions to avoid the spread of the many fungal, bacterial, and nematode pathogens that survive in old crop debris and in soil. These soil-borne pathogens can be carried into clean fields by the movement of workers or equipment. Improper irrigation and tillage can also create conditions which encourage the survival and spread of cucurbit pathogens which reside in soil.

Also difficult to control are diseases carried on seed, such as anthracnose, angular leafspot disease, and gummy stem blight. Control of these diseases requires pre-planting fungicide seed treatment. Chemical fungicide treatment is expensive, time-consuming, and potentially hazardous to the health of those who apply it.

The present invention is directed to overcoming these and other deficiencies in the art.

### SUMMARY OF THE INVENTION

The present invention relates to a multiple disease resistant inbred cucumber seed, a sample of said seed having been deposited under ATCC accession number PTA-4029.

The present invention also relates to a tissue culture of regenerable cells of an inbred cucumber plant, a representative seed of this inbred cucumber plant having been deposited under ATCC Accession No. PTA-4029, where such tissue regenerates plants capable of expressing all the morphological and physiological characteristics of an inbred cucumber plant.

The present invention also relates to a method for producing a multiple disease resistant inbred cucumber seed. This involves providing a first C. sativus L. cucumber inbred line which is resistant to target leafspot disease and a second C. sativus L. cucumber inbred line which is resistant to zucchini yellow mosaic virus, watermelon mosaic virus, and papaya ringspot virus. The first C. sativus L. cucumber line is crossed with the second C. sativus L. cucumber inbred line to yield an F1 hybrid C. sativus L. cucumber line which is resistant to target leafspot disease. The F1 hybrid C. sativus L. cucumber line is backcrossed to the second C. sativus L. cucumber inbred line yield a third C. sativus L. inbred cucumber line which is target leafspot disease, zucchini yellow mosaic virus, watermelon mosaic virus, and papaya ringspot virus resistant; and the seed is harvested therefrom.

The use of disease-resistant varieties is an economical means of controlling disease in vegetable crops. Therefore, the development of a variety of slicing cucumber with an improved scope of disease resistance is highly desirable. The present invention uses traditional plant breeding techniques to develop a highly disease resistant cucumber cultivar that is commercially appealing.

### BRIEF DESCRIPTION OF THE DRAWINGS

FIG. 1 is a diagram of the breeding history of the ‘Poinsett 97’ (also known as NYOO-823-N) cucumber inbred line. Key: “BC” stands for backcross; “P” stands for Poinsett; “PRV” stands for potyvirus resistance; “TLS” stands for target leafspot (Corynespora cassiicola); “TLSR” stands for target leafspot resistance; “SP” stands for self pollination. The exponent in P93 indicates the number of backcrosses to ‘P93.’

FIG. 2 is a photograph of a ‘Poinsett 97’ plant prior to fruiting.

FIG. 3 is a photograph of a ‘Poinsett 97’ fruit.

### DETAILED DESCRIPTION OF THE INVENTION

The following definitions apply to the present application.

The terms “cultivar” and “variety” are used interchangeably.

The term “backcrossing” refers to a process in which a breeder repeatedly crosses hybrid progeny, for example a first generation hybrid (F1), back to one of the parents of the hybrid progeny.

The term “crossing” refers to the mating of two parent plants.

The term “F1 hybrid” refers to the first generation progeny of the cross of two non-isogenic plants.

The term “genotype” refers to the genetic constitution of a cell or an organism.

The term “hybrid” refers to a plant that is an offspring derived by crossing two inbred lines. Seed from fruits produced from hybrid seed cannot be saved because two specific inbred lines must be used exclusively to obtain hybrid seed.

The term “inbred” refers to true-breeding cultivars that are capable of self pollination because the plants are monoecious, i.e., have male and female reproductive organs on separate flowers, but on the same individual.

The term “pistillate flowers” refers to female flowers that produce fruit.

The term “phenotype” refers to the detectable characteristics of a cell or organism, which characteristics are the manifestation of gene expression.

The term “plant” refers to whole plants, plant protoplasts, plant cells of a tissue culture from which cucumber plants can be regenerated, plant calli, plant clumps, and plant cells that are intact in plants or parts of plants, including leaves, pollen, embryos, roots, tip, anthers, flowers, and the like.

The term potyvirus resistance (“PVR”) refers to a combined resistance to the potyviruses zucchini yellow mosaic virus (“ZYMV”), watermelon mosaic virus, (“WMV”), and papaya ringspot virus (“PRSV”).

“Resistance” as used herein means resistance to a disease, and not necessarily to the pathogen that causes the disease.

The present invention relates to a multiple disease resistant inbred cucumber seed designated ‘Poinsett 97’/NYOO-823-N. A sample of this seed has been deposited under ATCC Accession No. PTA-4029.

The present invention also relates to a method for producing a multiple disease resistant inbred cucumber seed known as “Poinsett 97’ (“P97”), also designated NYOO-823-N, of which a seed sample has been deposited under ATCC Accession No. PTA-4029. This involves providing a first C. sativus L. cucumber inbred line which is resistant to target leafspot disease and a second C. sativus L. cucumber inbred line which is resistant to zucchini yellow mosaic virus, watermelon mosaic virus, and papaya ringspot virus. The first C. sativus L. cucumber line is crossed with the second C. sativus L. cucumber inbred line to yield an F1 hybrid C. sativus L. cucumber line which is resistant to target leafspot disease. The F1 hybrid C. sativus L. cucumber line is backcrossed to the second C. sativus L. cucumber inbred line yield a third C. sativus L. inbred cucumber line which is target leafspot disease, zucchini yellow mosaic virus, watermelon mosaic virus, and papaya ringspot virus resistant; and the seed is harvested therefrom.

In one aspect of the present invention, the first parent used in the initial cross to produce the inbred line ‘P97’ is a cucumber cultivar known to be resistant to target leafspot disease (“TLS”), while the second parent is a cucumber cultivar resistant to zucchini yellow mosaic virus, watermelon mosaic virus, and papaya ringspot virus (“PRV”). The first parental cultivar may also be resistant to cucumber scab, powdery mildew, downy mildew, angular leafspot disease, anthracnose, and cucumber mosaic virus in addition to target leafspot disease. The second parental cultivar may be resistant to cucumber scab, powdery mildew, downy mildew, angular leafspot disease, anthracnose, and cucumber mosaic virus in addition to zucchini yellow mosaic virus, watermelon mosaic virus, and papaya ringspot virus.

An example of a suitable first parental cultivar of the present invention is ‘Poinsett 87’ (“P87”), a slicing variety of C. sativus L. ‘P87’ is characterized as monoecious, with uniform color, bitter fruit, and having resistance to cucumber mosaic virus (“CMV”), cucumber scab, powdery and downy mildew, angular leafspot disease, anthracnose, TLS, and Ulocladium leafspot. (For a history of the pedigree of the ‘Poinsett’ cultivar, see Munger, H. M., “Breeding Multiple Viral Resistance in Cucurbits,” in Resistance to Viral Disease of Vegetables, pp. 44-60, M. Kyle, ed., Timber Press, Portland (1993), which is hereby incorporated by reference in its entirety.)

An example of a suitable second parental cultivar of the present invention is ‘Poinsett 93’ (“P93), a slicing variety of C. sativus. ‘P93’ is characterized as monoccious, with uniform color, bitter fruit, and resistance to cucumber scab, powdery mildew, downy mildew, angular leafspot disease, anthracnose, CMV, ZYMV, WMV, and PRSV (Munger, H. M. “Breeding Viral Resistance in Cucumbers,” in Resistance to Viral Diseases of Vegetables, pp. 44-60, Kyle, ed. Timber Press, Inc., Portland (1993), which is hereby incorporated by reference in its entirety).

The present invention also relates to a multiple disease resistant inbred cucumber plant that is grown from the multiple disease resistant inbred seed of the present invention, known as “Poinsett 97’, also designated NYOO-823-N, a sample of which seed having been deposited under ATCC Accession No. PTA-4029. Thus, the present invention further relates to an inbred cucumber plant which is resistant to cucumber scab, powdery mildew, downy mildew, angular leafspot disease, anthracnose, CMV, TLS, ZYMV, WMV, PRSV, Altemaria leaf blight, and Ulocladium leafspot disease.

The present invention also encompasses the use of germplasm from the inbred multiple disease resistant plant of the present invention in any breeding program to yield the disease resistant inbred cucumber seeds or plant of the present invention, and any inbred multiple disease resistant seeds, plants, and plant parts derived therefrom.

The present invention further relates to the inbred cucumber seed produced by the method of the present invention which is resistant to cucumber scab, powdery mildew, downy mildew, angular leafspot disease, anthracnose, CMV, TLS, ZYMV, WMV, PRSV, Alternaria leaf blight, and Ulocladium leafspot disease. After the disease resistant cultivar of the present invention has been tested and inbred individuals identified and selected for optimum performance, the inbred seed of the present invention is produced from the inbred disease resistant plant of the present invention.

From the inbred resistant seed, it is possible to grow an inbred disease resistant plant having all the physiological and morphological characteristics of the resistant inbred cucumber plant described herein. Multiple disease resistant inbred cucumber plants of the present invention can be readily produced from the multiple disease resistant inbred seed of the present invention using growing techniques and conditions known to those skilled in the art. The present invention also encompasses such plants.

The present invention also relates to parts of the multiple disease resistant inbred cucumber plant of the present invention, especially the pollen and ovule of such a plant, from which a cucumber scab, powdery mildew, downy mildew, angular leafspot disease, anthracnose, CMV, TLS, ZYMV, WMV, PRSV, Alternaria leaf blight, and Ulocladium leafspot disease resistant cucumber plant can be produced. This aspect of the present invention also relates to a tissue culture of regenerable cells of the multiple disease resistant inbred cucumber plant of the present invention, where the tissue can be regenerated by methods well-known in the art to regenerate a plant capable of expressing all the morphological and physiological characteristics of the multiple disease resistant inbred cucumber plant of the present invention. Included in this aspect of the present invention is a plant grown from such a tissue culture, and seeds derived from a plant grown from tissue culture. Multiple disease resistant C. sativus L. inbred plants, seeds, and plant parts made using the method of the present invention under any traditional breeding methods, including, without limitation, pedigree breeding, selfing, intercrossing, and backcrossing are also encompassed by the present invention.

Plant breeding programs are designed to produce new cultivars with superior qualities by the deliberate crossing of parent plants with the most highly desirable traits. Resistance to disease is a highly desirable trait, therefore, disease resistance is often one goal of a plant breeding program. How that goal is accomplished may vary, depending on the plant being bred. For example, in self-pollinated and many open pollinated crops, the development of disease-resistant varieties can routinely be accomplished with the backcross and pedigree methods. Backcrossing is especially useful for transferring a specific desirable trait from one inbred source to an inbred that lacks that trait. Backcross methods rapidly introgress specific resistance genes from a donor parent into a desirable variety, concurrently reconstructing the original variety. For example, this can be accomplished by first crossing a superior inbred (recurrent parent) to a donor inbred (non-recurrent parent), that carries the appropriate gene(s) for the trait in question. The recurrent parent typically includes many commercially important traits, while the non-recurrent parent includes a resistance trait, as is the case for the TLS resistant cucumbers. The progeny of this cross are then mated back to the superior recurrent parent followed by selection in the resultant progeny for the desired trait to be transferred from the non-recurrent parent. Through backcrossing, a new variety is produced quickly, requires minimal testing, and is nearly isogenic with the original. Backcross breeding has been used extensively for breeding disease-resistant cultivars (Nickell et al., “Registration of L84-5873 and L84-5932 Soybean Germplasm Lines Resistant to Brown Stem Rot,” Crop Sci. 32:835 (1992), which is hereby incorporated by reference in its entirety).

Also suitable for incorporating disease resistance are breeding methods such as pedigree, F2 selection, single seed descent, and the nested hierarchy method. Pedigree methods are useful when both parents are adapted and carry genes for resistance to different diseases. Compared with the backcross method, a more genetically diverse germplasm base is developed, but the time to release a variety is much longer. The backcross and pedigree methods are forms of inbreeding that ultimately result in the development of pure lines. The level of homozygosity increases with each generation of self- or backcrossing.

The selection of a suitable recurrent parent is an important step for a successful backcrossing procedure. The goal of a backcross is to alter or substitute a single trait or characteristic in the original inbred plant. This is accomplished by modifying or substituting a single gene of the recurrent inbred with the desired gene from the recurrent parent, while retaining essentially all of the rest of the desired physiological and morphological characteristics of the original inbred.

A breeding program in accordance with the present invention will include a periodic objective evaluation of the finished variety. Evaluation criteria include disease-resistance and other characteristics important in commercial cucumber crops, such as vigor, uniformity, earliness, and yield. Resistance as used herein means resistance to a disease, and not necessarily to the pathogen that causes the disease. Breeders select for resistance to disease on the basis of reduced symptoms and better growth, seldom knowing whether the ‘resistance’ is brought about by some mechanism such as reduced replication, or movement of the virus, or by tolerance to the presence of the virus. It is convenient to speak of resistance to cucumber mosaic virus, for example, but more accurate to talk about resistance to the disease. Following initial evaluations, promising advanced breeding lines are thoroughly tested and evaluated against appropriate standards in the commercial target area. The best lines are potential new commercial cultivars.

For a breeder interested in improving the performance of a genotype under field conditions, yield and quality of the marketable portion of the plant are of primary concern. Therefore, specific phenotypic traits that are considered “desirable” characteristics are selected for in commercial breeding programs. Incorporation of resistance to a single pathogen is accomplished via selection over one or more generations. In contrast, breeding for multiple pathogen resistance presents three other possible approaches to selection, including tandem selection, independent culling levels, and the selection index (Turner et al., Quantitative Genetics in Sheep Breeding, Cornell University Press, Ithaca, NY (1967), which is hereby incorporated by reference in its entirety). These techniques were developed primarily for quantitative traits and are used regularly in most plant and animal breeding programs; they also provide the theoretical foundation for the selection of qualitatively inherited traits. These principles are well-known to those skilled in the art.

In breeding for multiple resistances, the biology of the different pathogens and their interactions with the host species affects the choice of a selection technique. Cross protection, synergism, variations in symptomatology, and escapes influence selection for multiple pathogen resistance. For example, cross protection resulting from simultaneous inoculations of closely related viruses can confound identification of resistant and susceptible phenotypes, and affect the accuracy of selection. In addition, the interaction of different viruses with the different genetic backgrounds of the host may cause symptomatology to vary, becoming an unreliable criteria for selection. Synergism among pathogens may affect the ability to detect pathogenicity. Lastly, escapes affect all selection techniques, but can be detected with repeated inoculations within a generation or over successive generations. Those skilled in the art will appreciate how these selection methods and others can be applied to practice the present invention.

Several reference books are available that describe these and various other breeding methods suitable for producing the multiple disease resistant inbred and hybrid cucumber seeds and plants of the present invention, including, but not limited to, Allard, R. W., Principles of Plant Breeding, New York: Wiley (1960); Simmonds, N. W., ed., Evolution of Crop Plants, London: Longman (1979); Simmonds, N. W., ed., Principles of Crop Improvement, London, New York: Longman (1979); Sneep et al., eds., Plant Breeding Perspectives Wageningen, Neth.: Centre for Agricultural Publishing and Documentation (1979); and Fehr et al., eds., Principles of Cultivar Development, New York: Macmillan (1987), which are hereby incorporated by reference in their entirety. The information in the recommended texts, as well as those techniques known to the skilled artisan, can be used to carry out the present invention.

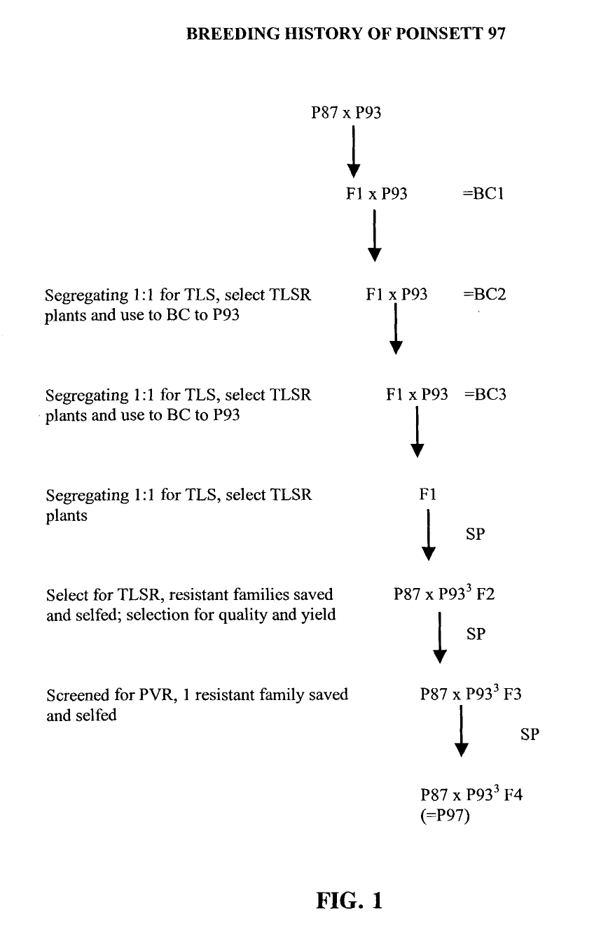

An example of a multiple disease resistant plant produced by the present invention is the cucumber cultivar designated ‘Poinsett 97.’ ‘Poinsett 97’ is the result of the cross between ‘Poinsett 87’ and ‘Poinsett 93’ (see Example 1 for details), and then backcrossing to ‘P93’ and selecting for TLS and PVR resistance. The breeding history of ‘P97’ is shown in FIG. 1. This yielded an exemplary cucumber variety of the present invention with resistance to cucumber scab, powdery mildew, downy mildew, angular leafspot disease, CMV, TLS, ZYMV, WMV, PRSV, Altemaria leaf blight, and Ulocladium leafspot disease. The cultivar ‘Poinsett 97’ is characterized by phenotype as shown in Table 1.

TABLE 1

| Characteristic | | Poinsett ‘97 Phenotype |
|---|---|---|
| Predominate usage | | Slicing, Fresh Market |
| Predominate culture | | Outdoor |
| Days from seeding to market maturity | | 53 days |
| Habit | | Vine |
| Growth type | | Indeterminate |
| Sex | | Monoecious |
| Length of main stem (average of 25 plants) | | 135 cm |
| Internode Length, main stem (average of 25 | | 11 cm |
| plants) | | |
| Number of Nodes from Cotyledon leaves to | | 3 |
| Node bearing the first pistillate flower | | |
| Surface of plant mainstem | | Grooved, Ridged |
| Leaf length (measurements from mature | | 203 mm |
| blade of third leaf; average of 25 plants) | | |
| Petiole length (measurements from mature | | 14 cm |
| blade of third leaf; average of 25 plants) | | |
| Petiole width (measurements from mature | | 231 mm |
| blade of third leaf; average of 25 plants) | | |
| Leaf color | | Medium green |
| Flower color | | Yellow |
| Fruit at Maturity: | Average Length | 21 cm |
| | Medial Diameter | 5 cm |
| | Average Weight | 250 gms |
| | Skin color | Predominately dark green at |
| | | both stem and blossom end; not |
| | | mottled; |
| | Stripes | Extend less than ⅓ of the fruit |
| | | length |
| | Skin texture | Thick, ribbed, dull, coarse |
| | Fruit shape | Not necked |
| | Stem end cross-section | Circular |
| | Medial cross section | Circular |
| | Blossom end cross-section | Circular |
| | Tubereles (warts) | Many, obscure |
| Fruit seed at harvest maturity: | Length | 25 cm |
| | Medial diam. | 6 cm |
| | Color | Yellow, not striped |
| | Surface | Smooth |
| | Netting | Slight or none |
| Fruit Setting | | Normally with Seeds |
| Seeds: | Average No. per fuit | 260 |
| | Gram per 1,000 seeds | 25 |

| | |
|---|---|
| Disease resistance: | Cucumber Scab (Cladosporium |
| | cucerinum); |
| | Powdery Mildew (Etysiphe |
| | chicoracearum; |
| | Downy Mildew; |
| | Angular Leafspot |
| | (Pseudomonas lachrymans); |
| | Anthracnose; |
| | Cucumber Mosaic Virus |
| | (CMV); |
| | Target Leafspot (Corynespora |
| | cassiicola); |
| | Zucchini Yellow Mosaic Virus |
| | (ZYMV); |
| | Watermelon mosaic virus |
| | (WMV); |
| | Papaya Ringspot Virus (PRSV); |
| | Altemaria Leaf Blight |
| | (Alternaria cucumerina); |
| | Ulocladium Leafspot |
| | (Ulocladium cucurbitae) |

The above description of the ‘P97’ cultivar in no way is meant to limit the phenotypic characteristics of the multiple disease resistant cucumber produced by the method of the present invention.

This invention also relates to the seeds of cucumber cultivar designated ‘P97’, to the plants of cucumber cultivar designated ‘P97’, and to methods for producing a cucumber plant produced by crossing the cucumber designated ‘P97’ with itself or another cucumber variety. All plants produced using the cucumber cultivar designated ‘Poinsett 97’ (NY-00-823-N) as a parent are within the scope of this invention. Advantageously, the cucumber variety could be used in crosses with other, different, cucumber plants to produce first generation (F1) cucumber hybrid seeds and plants with superior characteristics. Thus, any such methods using the cucumber cultivar of the present invention are encompassed by this invention, including, but not limited to, pedigree breeding, selfing, intercrossing, backcrossing, crosses to populations, and the like. This aspect of the present invention also relates to any plant, or parts thereof, produced by growing a plant resulting from a cross between NYOO-823-N and another, different cucumber plant, and the seeds derived from a plant resulting from such a cross.

### EXAMPLES

### Example 1

### Origin and Breeding of ‘Poinsett 97’

The exemplary multiple disease resistant inbred cultivar of the present invention, ‘Poinsett 97’ (“P97”), is the result of combining the resistance to target leafspot disease (“TLS”) of ‘Poinsett 87’ (“P87”) with the resistance to zucchini yellow mosaic virus (“ZYMV”), watermelon mosaic virus (“WMV”), and papaya ringspot virus (“PRSV”) of ‘Poinsett 93’ (“P93”), both parents having resistance to cucumber mosaic virus (“CMV”), scab, powdery mildew, and downy mildew. Both parents were derived from long-term on-going backcross breeding programs at Cornell University, Ithaca, N.Y., and are characterized as near-isogenic lines with ‘Poinsett’ background genotypes. The original ‘Poinsett’ cultivar, from which both ‘Poinsett 83’ and ‘Poinsett 97’ derive, was resistant to powdery and downy mildew, anthracnose, and angular leafspot (Barnes, W. C. “Multiple Disease Resistant Cucumbers,” Proc. Amer. Soc. Hort. Sci. 77:417-423 (1961), which is hereby incorporated by reference in its entirety). FIG. 1 is a diagram of the breeding history of the ‘Poinsett 97’ (also known as NYOO-823-N) cucumber inbred line.

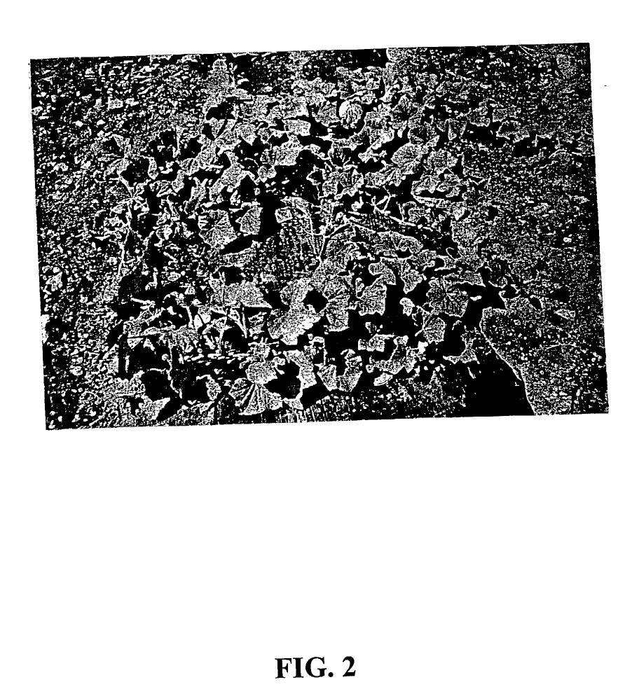

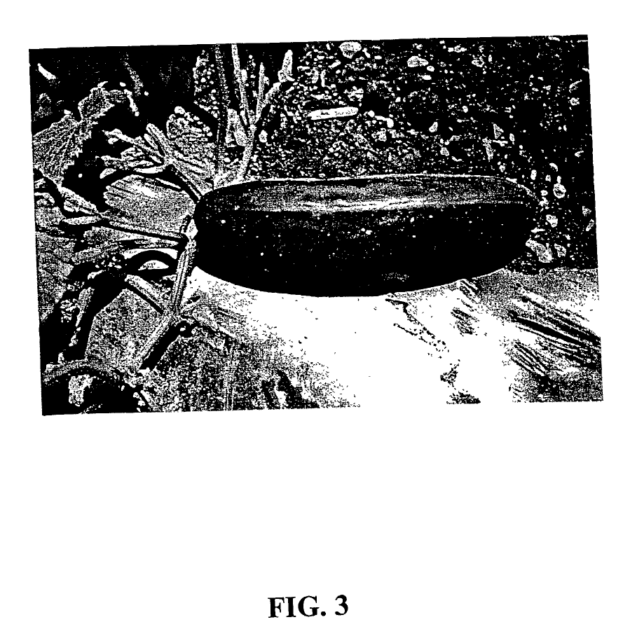

‘P87’ was crossed to ‘P93,’ producing an F1 hybrid resistant to TLS and susceptible to the three potyviruses ZYMV, WMV, and PRSV (these resistances are monogenically recessive). The F1 hybrid was then backcrossed three times to the parental cultivar ‘P93’, while selecting for TLS resistance. Out of a number of F3 progenies, 12 were found to be homozygous for TLS resistance. Based on the backcross breeding method being used, these were assumed to be carrying potyvirus resistance. These 12 lines were evaluated for yield and horticultural type. One line, 95-227-13, was chosen as slightly superior, but it was discovered that this line lacked potyvirus resistance. Apparently, there was an unfavorable linkage between TLS resistance and potyvirus susceptibility that had to be broken. Therefore, the original 12 lines carrying TLS resistance were evaluated for potyvirus resistance. Only one line, 95-227-12, was found to have retained resistance to the 3 potyviruses. Results showed 95-227-12 was homozygous for potyvirus resistance. There is not much difference in yield or type between 95-227-12 and 95-227-13. In addition, no significant variation has been observed within ‘P97’. ‘P97’ has been shown to be genetically stable, having remained unchanged in its disease resistances through several generations. No regular classes or variants are regularly observed during reproduction and multiplication, although the yield may vary depending on cultivation conditions. FIG. 2 is a photograph of a ‘Poinsett 97’ plant, prior to fruiting. FIG. 3 is a photograph of a ‘Poinsett 97’ fruit.

### Example 2

### Distinctiveness of ‘Poinsett 97’

‘Poinsett 97’ differs substantially from the earlier ‘Poinsett’ variety by having resistance to eight additional diseases. ‘Poinsett’ was bred at Clemson Station in Charleston, S.C., and was resistant to powdery mildew, downy mildew, anthracnose, and angular leafspot disease (Barnes, W. C. “Multiple Disease Resistant Cucumbers,” Proc. Amer. Soc. Hort. Sci. 77:417-423 (1961); Munger, H. M. “Breeding Viral Resistance in Cucumbers,” in Resistance to Viral Diseases of Vegetables, pp. 44-60, Kyle, ed. Timber Press, Inc., Portland (1993), which are hereby incorporated by reference in their entirety).

‘Poinsett 97’ retains these resistances, and adds resistance to CMV, the potyviruses ZYMV, WMV, PRSV, cucumber scab, and target leafspot disease. Also, field tests indicate that ‘Poinsett 97’ has higher powdery mildew and downy mildew resistance than ‘Poinsett.’ In addition, cucumbers resistant to TLS are also expected to be resistant to Ulocladium (Zitter, et al., “A Leaf Spot of Cucumber Caused by Ulocladium cucurbitae in New York,” Plant Dis. 74:824-827 (1990), which is hereby incorporated by reference in its entirety). Resistance to Alternaria in cucumbers carrying TLS resistance has also been seen in the test plots. Lastly, ‘P97’ has uniform color, which the original ‘Poinsett’ does not have.

‘Poinsett 97’ differs from the immediate parental cultivar ‘Poinsett 87’ by having resistance to ZYMV, WMV, and PRSV. ‘Poinsett 97’ also exhibits a high level of CMV resistance, in comparison to a rating of medium-high CMV resistance for ‘Poinsett 87.’

‘Poinsett 97’ differs from the immediate parental cultivar ‘Poinsett 93’ by having resistance to TLS which ‘Poinsett 93’ does not have.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A multiple disease resistant inbred cucumber seed, a sample of said seed having been deposited under ATCC Accession No. PTA-4029.

2. A multiple disease resistant cucumber plant, or parts thereof, produced by growing the seed of claim 1.

3. Pollen of the plant according to claim 2.

4. An ovule of the plant according to claim 2.

5. A tissue culture of regenerable cells of an inbred cucumber plant, representative seed of said inbred cucumber plant having been deposited under ATCC Accession No. PTA-4029, wherein the tissue regenerates plants capable of expressing all the morphological and physiological characteristics of an inbred cucumber plant.

6. A tissue culture according to claim 5, wherein the tissue culture is produced from cells or protoplasts from a plant part selected from the group consisting of leaves, pollen, embryos, roots, tip, anthers, and flowers.

7. A cucumber plant regenerated from the tissue culture according to claim 5.

8. A method for producing a multiple disease resistant inbred cucumber seed comprising:
providing a first C. sativus L. cucumber inbred line which is resistant to target leafspot disease;
providing a second C. sativus L. cucumber inbred line which is resistant to zucchini yellow mosaic virus, watermelon mosaic virus, and papaya ringspot virus;
crossing the first C. sativus L. cucumber line with the second C. sativus L. cucumber line to yield an F1 hybrid C. sativus L. cucumber line which is resistant to target leafspot disease;
backcrossing the F1 hybrid C. sativus L. cucumber line to the second C sativus L. cucumber inbred line to yield a third C. sativus L. inbred cucumber line which is target leafspot disease, zucchini yellow mosaic virus, watermelon mosaic virus, and papaya ringspot virus resistant; and
harvesting the seed therefrom.

9. The method according to claim 8, wherein the first inbred cucumber line is additionally resistant to cucumber scab, powdery mildew, downy mildew, angular leafspot disease, anthracnose, and cucumber mosaic virus.

10. The method according to claim 8, wherein the second inbred cucumber line is additionally resistant to cucumber scab, powdery mildew, downy mildew, angular leafspot disease, anthracnose, and cucumber mosaic virus.

11. The method according to claim 8, wherein the first inbred cucumber line is the ‘Poinsett 87’ cultivar.

12. The method according to claim 8, wherein the second inbred cucumber line is the ‘Poinsett 93’ cultivar.

13. A method according to claim 8, further comprising:
growing the inbred seed to yield a cucumber inbred plant which is resistant to target leafspot disease, zucchini yellow mosaic virus, watermelon mosaic virus, and papaya ringspot virus.

14. The method according to claim 8, further comprising:
using germplasm derived from the third cucumber inbred line in a plant breeding program to yield disease resistant cucumber inbred seeds, wherein said breeding program comprises traditional plant breeding techniques selected from the group consisting of pedigree breeding, selfing, intercrossing, and backcrossing.

15. The method according to claim 14, wherein the third cucumber inbred line is additionally resistant to cucumber scab, powdery mildew, downy mildew, angular leafspot disease, anthracnose, cucumber mosaic virus, target leafspot disease, zucchini yellow mosaic virus, watermelon mosaic virus, papaya ringspot virus, Alternaria leaf bight, and Ulocladium leafspot disease.

16. A multiple disease resistant inbred cucumber seed produced by the method according to claim 8.

17. A multiple disease resistant inbred cucumber plant, or parts thereof, produced by the method according to claim 8.

18. Pollen of the plant according to claim 17.

19. An ovule of the plant according to claim 17.

20. A method for developing a cucumber plant comprising:
subjecting the cucumber plant, or parts thereof, according to claim 2 to a plant breeding program.

21. A method according to claim 21, wherein the plant breeding program comprises techniques selected from the group consisting of pedigree breeding, selfing, intercrossing, and backcrossing.

22. A cucumber plant, or parts thereof, produced by the method of claim 20.

23. A method of producing a hybrid cucumber seed comprising:
crossing an inbred cucumber plant according to claim 2 with another, different cucumber plant.

24. A hybrid cucumber seed produced by the method of claim 23.

25. A hybrid cucumber plant, or parts thereof, produced by growing the hybrid cucumber seed according to claim 24.

26. Cucumber seed produced by growing said hybrid plant according to claim 24 and harvesting cucumber seeds from that plant.
